(19) United States
(12) Patent Application Publication
(10) Pub. No.: US 2017/0259024 A1
(43) Pub. Date: Sep. 14, 2017

(54) SMALL ANIMAL ANESTHESIA SYSTEM

(71) Applicant: YU-PIN-TANG TRADITIONAL CHINESE MEDICINE FOUNDATION, Kaohsiung City (TW)

(72) Inventor: Young-Bin CHEN, Kaohsiung City (TW)

(21) Appl. No.: 15/064,009

(22) Filed: Mar. 8, 2016

(51) Int. Cl. A61M 16/10; A61M 16/06; A61M 16/08; A61M 16/00

(52) U.S. Cl. CPC A61M 16/104; A61M 16/0063; A61M 16/06; A61M 16/0875; A61M 16/105; A61M 2250/00; A61M 2230/005

(57) ABSTRACT

A small animal anesthesia system is provided, including a compressor, an anesthetizing gas generator and an anesthesia platform. The compressor has a first outlet and a first intake. The anesthetizing gas generator has a second outlet and a second intake. The first outlet of the compressor is connected to the second intake of the anesthetizing gas generator to supply gas, so that an anesthetizing gas is generated. The anesthesia platform includes a bearing plate, an anesthetic mask and a first anesthesia chamber. The anesthetic mask is configured to cover the face of the small animal, and connected to the second outlet of the anesthetizing gas generator, such that the small animal is anesthetized by the anesthetizing gas generated by the anesthetizing gas generator. The first anesthesia chamber is disposed on the bearing plate, and connected to the first intake of the compressor to recycle the anesthetizing gas.

### BACKGROUND OF THE INVENTION

1. Field of the Invention

This application relates to an anesthesia system, and more particularly, to a small animal anesthesia system which is feasible to anesthetize small animals.

2. Description Of the Related Art

Conventionally, anesthetizing small animals such as rats has been widely applied in the biomedicals, pre-clinical testings and teaching experiments. The purpose of anesthetizing small animals is not only to alleviate pain to the small animals and meet the animal testing regulations, but also to ensure that the surgery to the small animals and the experimental research can be performed successfully.

Generally speaking, the small animals are anesthetized by an anesthetizing gas while being experimented. When it comes to the inhalation of anesthetizing gas, it has to dilute the anesthetizing gas adequately to prevent the operation personnel from being affected by the anesthetizing gas in the process of anesthetizing the small animal. As a rule, the anesthetizing gas is diluted by activated carbons, but such manner is incapable of using the anesthetizing gas effectively.

As a consequence, the inventor of the present disclosure who has been mulling over the technical problems mentioned above, designs a small animal anesthesia system aiming at improving the current shortcomings, so as to promote the industrial practicability.

### SUMMARY OF THE INVENTION

In view of the aforementioned technical problems, the primary objective of the present disclosure provides a small animal anesthesia system to resolve the pending technical problems.

According to the objective of the present disclosure, a small animal anesthesia system is provided, which includes a compressor, an anesthetizing gas generator and an anesthesia platform. The compressor is configured to generate gas, and disposed with a first outlet and a first intake. The anesthetizing gas generator is configured to generate an anesthetizing gas, and disposed with a second outlet and a second intake. The first outlet of the compressor is connected to the second intake of the anesthetizing gas generator via a first gas supplying tube to supply a gas to the anesthetizing gas generator to generate the anesthetizing gas. An anesthesia platform includes a bearing plate, an anesthetic mask and a first anesthesia chamber. The anesthetic mask is a mask structure and configured to cover face of the small animal. The anesthetic mask is connected to the second outlet of the anesthetizing gas generator via a second gas supplying tube. The first anesthesia chamber is disposed on the bearing plate and has an opening. The first anesthesia chamber is connected to the first intake of the compressor via a recycling tube. Here, the small animal covered by the anesthetic mask is placed in the first anesthesia chamber, and the second gas supplying tube is configured to pass through the opening to guide the anesthetizing gas generated by the anesthetizing gas generator to anesthetize the small animal.

Preferably, the bearing plate is disposed with a retaining base, and the anesthetic mask has a retaining head. When the small animal's face is covered by the anesthetic mask, the retaining base and the retaining head are magnetically attached to each other so as to restrain the small animal.

Preferably, the retaining head is disposed at one side of the anesthetic mask.

Preferably, an edge of the opening of the first anesthesia chamber is disposed with a leak-proof edge to tightly clamp the second gas supplying tube.

Preferably, the small animal anesthesia system further includes a second anesthesia chamber, a distributor disposed on the second gas supplying tube, and a third gas supplying tube connected to the distributor to supply the anesthetizing gas to the second anesthesia chamber.

Preferably, the first anesthesia chamber is configured to cover head of the small animal, and the second anesthesia chamber is configured to accommodate the entire small animal.

Preferably, the small animal anesthesia system further includes a controller connected to the anesthetizing gas generator and a weight sensor disposed on the bearing plate to measure weight of the small animal. The controller adjusts a concentration of the anesthetizing gas generated by the anesthetizing gas generator according to the weight measured.

Preferably, the small animal anesthesia system further includes an activated carbon device disposed between the first anesthesia chamber and the first intake of the compressor.

As mentioned previously, a small animal anesthesia system provided in the present disclosure may have one or more advantages as follows.

1. A small animal anesthesia system of the present disclosure circularly recycles the anesthetizing gas to the compressor, and then the recycled anesthetizing gas is supplied to the anesthetizing gas generator, so that the anesthetizing gas can be used economically to reduce unnecessary waste.

2. A small animal anesthesia system of the present disclosure is disposed with the retaining base and retaining head which are attached to each other magnetically, so that the small animal can be restrained tightly while remaining adjustable.

3. The first anesthesia chamber of a small animal anesthesia system of the present disclosure is disposed with an opening which has a leak-proof edge to further prevent the leakage of the anesthetizing gas, preventing the anesthetizing gas from affecting the operation personnel and to increase the recovery efficiency of the anesthetizing gas.

4. The second anesthesia chamber of a small animal anesthesia system of the present disclosure is connected to the distributor, such that the anesthetizing gas is distributed to anesthetize the small animal in advance. Consequently, the usability of the small animal anesthesia system is promoted.

5. A small animal anesthesia system of the present disclosure is disposed with the weight sensor and the controller, so that the concentration of anesthetizing gas can be adjusted manually or automatically, so as to improve the convenience.

### BRIEF DESCRIPTION OF THE DRAWINGS

FIG. 1 is a block diagram of the first embodiment of a small animal anesthesia system of the present disclosure.

FIG. 2. is a schematic diagram of the first embodiment of a small animal anesthesia system of the present disclosure.

FIG. 3 is a schematic diagram of the second embodiment of a small animal anesthesia system of the present disclosure.

FIG. 4 is a block diagram of the third embodiment of a small animal anesthesia system of the present disclosure.

FIG. 5 is a block diagram of the fourth embodiment of a small animal anesthesia system of the present disclosure.

### DETAILED DESCRIPTION OF THE PREFERRED EMBODIMENTS

Reference will now be made in detail to the exemplary embodiments of the present disclosure, examples of which are illustrated in the accompanying drawings. Therefore, it is to be understood that the foregoing is illustrative of exemplary embodiments and is not to be construed as being limited to the specific embodiments disclosed, and that modifications to the disclosed exemplary embodiments, as well as other exemplary embodiments, are intended to be included within the scope of the appended claims. These embodiments are provided so that this disclosure will be thorough and complete, and will fully convey the inventive concept to those skilled in the art. The relative proportions and ratios of elements in the drawings may be exaggerated or diminished in size for the sake of clarity and convenience in the drawings, and such arbitrary proportions are only illustrative and not limiting in any way. The same reference numbers are used in the drawings and the description to refer to the same or like parts.

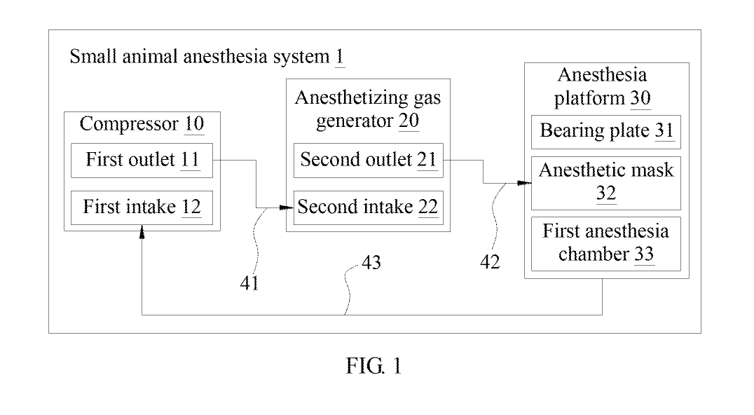

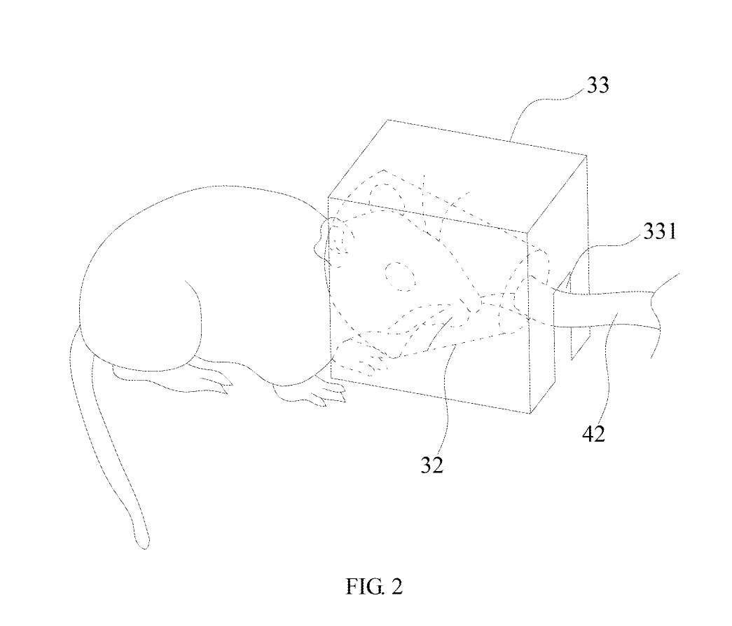

Please refer to FIG. 1 and FIG. 2 together, which are a block diagram of the first embodiment of a small animal anesthesia system of the present disclosure and a schematic diagram of the first embodiment of a small animal anesthesia system 1 of the present disclosure, respectively. As shown in the figures, a small animal anesthesia system 1 of the present disclosure is applied to anesthetize small animals such as rats. The small animal anesthesia system 1 includes a compressor 10, an anesthetizing gas generator 20, an anesthesia platform 30 and a plurality of connecting tubes. Here, the connecting tubes include a first gas supplying tube 41, a second gas supplying tube 42 and a recycling tube 43.

To be precise, the compressor 10 is disposed with a first outlet 11 and a first intake 12. The anesthetizing gas generator 20 is disposed with a second outlet 21 and a second intake 22. Here, the first outlet 11 of the compressor 10 is connected to the second intake 22 of the anesthetizing gas generator 20 via the first gas supplying tube 41, such that the gas generated by the compressor 10 is supplied to the anesthetizing gas generator 20 for the generation of anesthetizing gas.

Regarding the connection mode among the anesthesia platform 30, the compressor 10 and the anesthetizing gas generator 20. The anesthesia platform 30 includes a bearing plate 31, an anesthetic mask 32 and a first anesthesia chamber 33. The anesthetic mask 32 is a mask structure and configured to cover the face of the small animal. The anesthetic mask 32 is connected to the second outlet 21 of the anesthetizing gas generator 20 via the second gas supplying tube 42. Hence, the anesthetizing gas generated by the anesthetizing gas generator 20 is supplied to the face of the small animal and then the small animal is anesthetized after inhaling the anesthetizing gas. The first anesthesia chamber 33 is connected to the first intake 12 of the compressor 10 via the recycling tube 43, such that the anesthetizing gas which overflows from the anesthetic mask 32 to the first anesthesia chamber 33 is recycled and then reused by the compressor 10.

In practice, the small animal covered by the anesthetic mask 32 is placed on the bearing plate 31, and then the first anesthesia chamber 33 covers the small animal covered by the anesthetic mask 32 to prevent the leakage of the anesthetizing gas and to recycle the anesthetizing gas. Here, the first anesthesia chamber 33 is disposed with an opening 331, and the second gas supplying tube 42 is configured to pass through the opening 331 to enter the first anesthesia chamber 33. It is worth mentioning that an edge of the opening 331 is disposed with a leak-proof edge which can be an elastic band or a rubber strip. The leak-proof edge is applied to adequately clamp or attach to the second gas supplying tube 42 so as to further reduce the leakage of the anesthetizing gas.

As mentioned previously, because each member of the present disclosure is connected in the form of a circulation, it can effectively apply the anesthetizing gas so as to reduce the unnecessary waste.

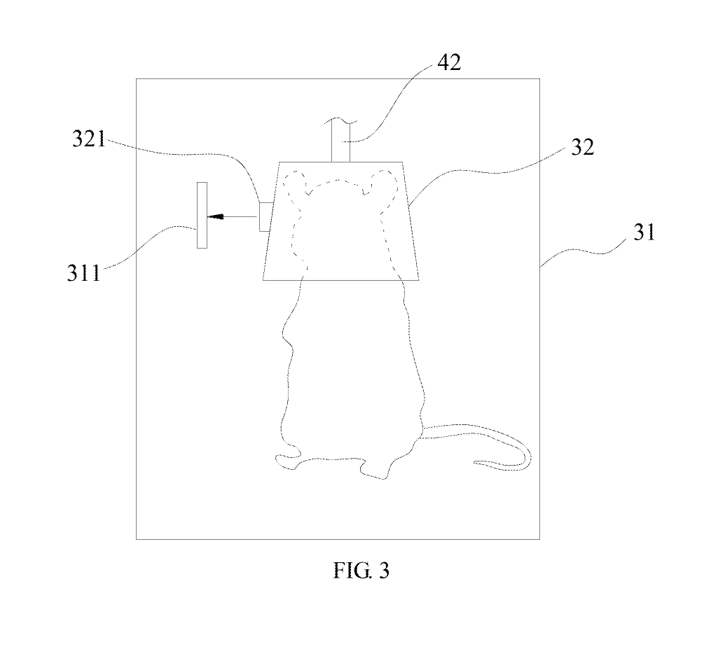

Please refer to FIG. 3, which is a schematic diagram of the second embodiment of a small animal anesthesia system 1 of the present disclosure. Members with the same reference numeral and disposition thereof applied in the present embodiment are the same as that of the former embodiment, and thus, the unnecessary details are no longer given herein. The difference between the former embodiment and the present embodiment lies that a retaining structure of the bearing plate 31 is provided in the present embodiment.

FIG. 3 is a top view of the bearing plate 31. As shown in the figure, a retaining base 311 is disposed on the bearing plate 31. A retaining head 321 is disposed on the anesthetic mask 32. Either one or both of the retaining base 311 and the retaining head 321 is disposed with a magnetic member, so that the retaining base 311 and the retaining head 321 are attached to each other by the magnetic member. Since the retaining base 311 and the retaining head 321 are attached to each other magnetically, the operation personnel can easily adjust the status of the small animal covered by the anesthetic mask 32 on the bearing plate 31 such as the small animal's pose, angle or position of the small animal's head, and so on. Preferably, the retaining head 321 is disposed at one side of the anesthetic mask 32 to facilitate the operation personnel in adjusting the status of the small animal covered by the anesthetic mask 32 on the bearing plate 31.

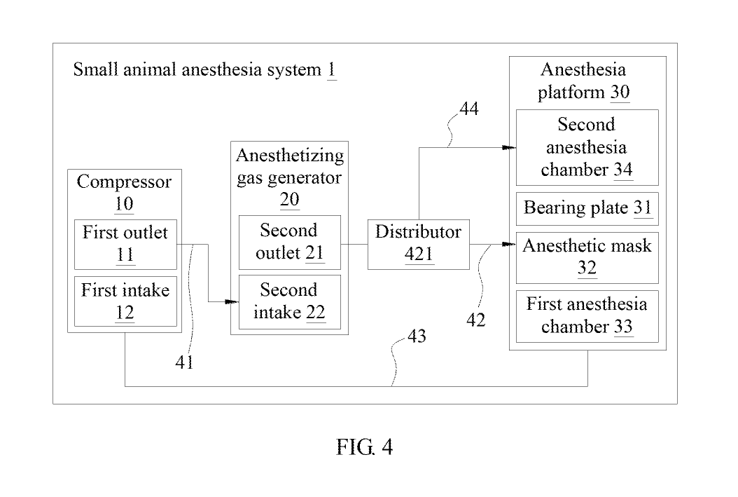

Please refer to FIG. 4, which is a block diagram of the third embodiment of a small animal anesthesia system 1 of the present disclosure. Members with the same reference numeral and disposition thereof applied in the present embodiment are the same as that of the former embodiment, and thus, the unnecessary details are no longer given herein. The difference between the former embodiment and the present embodiment lies that a second anesthesia chamber 34 is provided ill the present embodiment to anesthetize the small animal in advance.

As shown in the figure, the second gas supplying tube 42 connected between the anesthetic mask 32 and the second outlet 21 of the anesthetizing gas generator 20 is disposed with a distributor 421 which may be a combination of a three-way valve and a valve switch. A third gas supplying tube 44 is connected to the distributor 421. That is, the third gas supplying tube 44 is connected between the second anesthesia chamber 34 and the distributor 421, enabling the anesthetizing gas generated by the anesthetizing gas generator 20 to be supplied to the second anesthesia chamber 34.

In practice, the operation personnel anesthetize the small animal in advance. Here, the small animal is placed in the second anesthesia chamber 34 entirely, and the distributor 421 is selected to distribute the anesthetizing gas to the second anesthesia chamber 34 without distributing to the anesthetic mask 32. As preanesthesia has been performed to the small animal, the small animal's reaction is slowed. When the preanesthesia is completed, the small animal is taken out of the second anesthesia chamber 34 and covered by the anesthetic mask 32. Then the small animal is restrained on the bearing plate 31, and the first anesthesia chamber 33 covers the small animal's head. Afterwards, the distributor 421 distributes the anesthetizing gas to the anesthetic mask 32 while stopping the anesthetizing gas flow to the second anesthesia chamber 34 to anesthetize the small animal.

Therefore, it can be found in the present embodiment that the second anesthesia chamber 34 connected to the distributor 421 performs the preanesthesia to the small animal, thereby improving the functionality of the small animal anesthesia system 1 of the present disclosure.

It is noteworthy that when the anesthesia is about to complete, the operation personnel can dispose an activated carbon device (not shown in the figures) between the first anesthesia chamber 33 and the first intake 12 of the compressor 10, so that the anesthetizing gas can be diluted by the activated carbon device to prevent surplus anesthetizing gas from affecting the operation personnel.

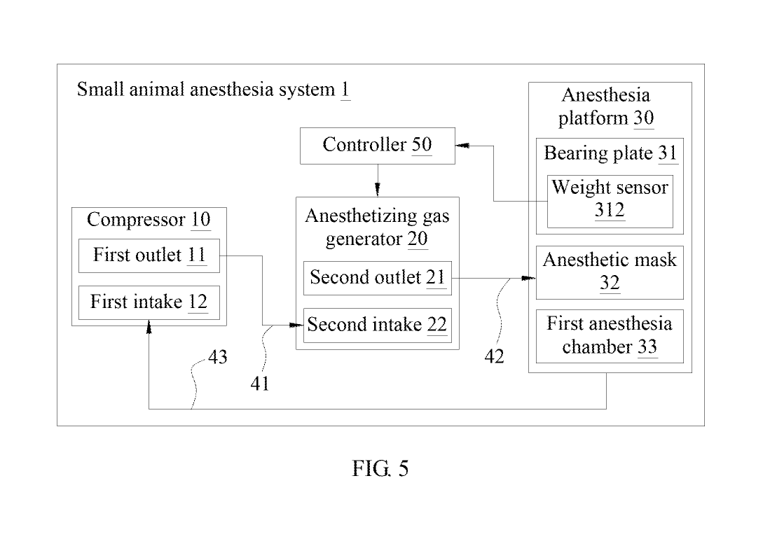

Please refer to FIG. 5, which is a block diagram of the fourth embodiment of a small animal anesthesia system 1 of the present disclosure. Members with the same reference numeral and disposition thereof applied in the present embodiment are the same as that of the former embodiment, and thus, the unnecessary details are no longer given herein. The difference between the former embodiment and the present embodiment lies that automatic components are provided in the present embodiment to enable the animal anesthesia system of the present disclosure to automatically adjust a concentration of the anesthetizing gas.

As shown in the figure, the animal anesthesia system 1 further includes a controller 50 electrically connected to the anesthetizing gas generator 20, and a weight sensor 312 is disposed on the bearing plate 31 and electrically connected to the controller 50. When the small animal is placed on the bearing plate 31, the weight sensor 312 measures a weight of the small animal, and then transmits the obtained weight to the controller 50. Afterwards, the controller 50 controls the anesthetizing gas generator 20 to generate a concentration of the anesthetizing gas corresponding to the weight of the small animal to anesthetize the small animal.

The controller 50 mentioned above may be an integrated circuit chip or a device disposed with an integrated circuit chip. The controller 50 can process signals through the integrated logical circuit or command in the form of software. The obtained weight will be translated into instructions by a controller 50 which facilitates the anesthetizing gas generator 20 to generate the concentration of the anesthetizing gas corresponding to the weight of the small animal to anesthetize the small animal.

As mentioned previously, the concentration of the anesthetizing gas is able to be adjusted automatically by the controller 50 and the weight sensor 312 applied the present embodiment, under the condition that excessive anesthetizing gas damaging the small animal can be avoided, such that the usability towards the operation personnel is promoted.

While the means of specific embodiments in present disclosure has been described by reference drawings, numerous modifications and variations could be made thereto by those skilled in the art without departing from the scope and spirit of the disclosure set forth in the claims. The modifications and variations should in a range limited by the specification of the present disclosure.

What is claimed is:

1. A small animal anesthesia system, comprising:
a compressor configured to generate a gas, and disposed with a first intake and a first outlet;
an anesthetizing gas generator configured to generate an anesthetizing gas, and disposed with a second intake and a second outlet, the first outlet of the compressor connected to the second intake of the anesthetizing gas generator via a first gas supplying tube to supply the gas to the anesthetizing gas generator, so as to generate the anesthetizing gas; and
an anesthesia platform, comprising:
a bearing plate;
an anesthetic mask which is a mask structure and configured to cover face of a small animal, the anesthetic mask connected to the second outlet of the anesthetizing gas generator via a second gas supplying tube; and
a first anesthesia chamber disposed on the bearing plate and having an opening, and the first anesthesia chamber connected to the first intake of the compressor via a recycling tube;
wherein, the small animal covered by the anesthetic mask is placed in the first anesthesia chamber, and the second gas supplying tube is configured to pass through the opening so as to anesthetize the small animal by the anesthetizing gas generated by the anesthetizing gas generator.

2. The small animal anesthesia system of claim 1, wherein the bearing plate is disposed with a retaining base, the anesthetic mask has a retaining head, and when the small animal is covered by the anesthetic mask, the retaining base and the retaining head are magnetically attached to each other so as to restrain the small animal.

3. The small animal anesthesia system of claim 2, wherein the retaining head is disposed at one side of the anesthetic mask.

4. The small animal anesthesia system of claim 1, wherein an edge of the opening of the first anesthesia chamber is disposed with a leak-proof edge to tightly clamp the second gas supplying tube.

5. The small animal anesthesia system of claim 1, further comprising a second anesthesia chamber, a distributor disposed on the second gas supplying tube, and a third gas supplying tube connected to the distributor to supply the anesthetizing gas to the second anesthesia chamber.

6. The small animal anesthesia system of claim 1, wherein the first anesthesia chamber is configured to cover head of the small animal, and the second anesthesia chamber is configured to accommodate the entire small animal.

7. The small animal anesthesia system of claim 1, further comprising a controller connected to the anesthetizing gas generator and a weight sensor disposed on the bearing plate to measure a weight of the small animal, and the controller adjusts a concentration of the anesthetizing gas generated by the anesthetizing gas generator according to the weight measured.

8. The small animal anesthesia system of claim 1, further comprising an activated carbon device disposed between the first anesthesia chamber and the first intake of the compressor.
